(19) United States
(12) Patent Application Publication
(10) Pub. No.: US 2005/0004504 A1
(43) Pub. Date: Jan. 6, 2005

(54) Catheter for extracorporeal treatment

(76) Inventors: Mark R. Frye, Blooming, IN (US); Bruce Gingles, Bloomington, IN (US); Thomas A. Osborne, Bloomington, IN (US); Jon D. Speer, Martinsville, IN (US)

Correspondence Address: INDIANAPOLIS OFFICE 27879;BRINKS HOFER GILSON & LIONE, ONE INDIANA SQUARE, SUITE 1600, INDIANAPOLIS, IN, 46204-2033, US

(21) Appl. No.: 10/874,409

(22) Filed: Jun. 23, 2004

Related U.S. Application Data
(60) Provisional application No. 60/482,149, filed on Jun. 24, 2003.

(51) Int. Cl. A61M 37/00

(52) U.S. Cl. 604/6.16; 604/4.01

(57) ABSTRACT

A catheter assembly for use in extracorporeal treatment of a bodily fluid. The catheter assembly includes a catheter body having an outer tubular member and an inner tubular member. At least one of the tubular members includes an expansion member, such as a malecot, for centering the catheter assembly in a body vessel. Body fluid is withdrawn from the vessel, and passed through one of the tubular members to a treatment instrument, such as a dialyzer. Treated fluid from the treatment instrument is then passed through the other tubular member and returned to the body vessel through one or more openings in said other tubular member.

### RELATED APPLICATIONS

The present patent document claims the benefit of the filing date under 35 U.S.C. §119(e) of Provisional U.S. patent application Ser. No. 60/482,149, filed Jun. 24, 2003, which is hereby incorporated by reference.

### BACKGROUND

1. Technical Field

The present application relates generally to a multi-lumen catheter for use in the extracorporeal treatment of bodily fluids, and more particularly, to a dual lumen catheter for use in a hemodialysis procedure.

2. Background Information

Catheters used in extracorporeal treatments of bodily fluids generally are provided with separate ingress and egress lines for transport of the bodily fluid to and from the patient. A bodily fluid, such as blood, is withdrawn from the body through one of the lines, generally referred to as the withdrawal line. The fluid is subjected to a treatment process and thereafter returned to the body through the other line, generally referred to as the infusion line.

With specific reference to the use of such catheters in a hemodialysis procedure, blood is withdrawn from the body through the withdrawal line of the catheter, passed through a dialyzer for treatment, and returned to the body through the infusion line. When such catheters are used in a hemodialysis procedure, they are generally inserted into the body through a major vein, such as the jugular vein, subclavian vein or the femoral vein. In addition to hemodialysis, extracorporeal catheters are also used for other medical procedures wherein a body fluid is removed from the body and thereafter returned following certain prescribed activity, such as pheresis and hemofiltration.

One problem with existing extracorporeal catheters is that such catheters can experience decreased flow rates over time. Decreased flow rates may be caused by, among other things, blockage of the withdrawal and/or infusion ports in the catheter. Various factors can cause a port to become blocked. Probably the most common causes of port blockage are the inadvertent positioning of one or more ports of the catheter against the vessel wall, thereby preventing or at least hindering free flow of fluid through the obstructed port, and the formation of fibrin sheaths that may occur along the ports in response to the vessel wall washing effect or clotting.

Several attempts have been made in the past to reduce port blockage. One method has been to provide multiple side ports that are spaced at various locations along the length of the catheter. The presence of multiple side ports, rather than a single port, reduces the effect of blockage of a single port. However, when multiple side ports are present, even these ports are still subject to blockage when placed against the vessel wall, or as a result of fibrin formation on the port. Other attempts have been made to reduce port blockage by providing a side-by-side dual lumen design having a stepped feature, wherein the respective withdrawal and infusion tubes are of different lengths. With this configuration, the ports withdraw and infuse the bodily fluid at different axial locations of the catheter. This arrangement may avoid some problems involved in maintaining adequate flow through the lumens, however such ports can still become blocked and be subject to suboptimal flow issues.

It is desired to provide a multi-lumen catheter for extracorporeal treatment of bodily fluids that minimizes port blockage, and that optimizes the flow of fluids through the lumens of the catheter.

### BRIEF SUMMARY

The present invention addresses the problems of the prior art by providing a multi-lumen catheter for extracorporeal treatment of bodily fluids, such as blood.

In one embodiment, the invention comprises a catheter assembly for use in extracorporeal treatment of a bodily fluid. The catheter assembly comprises a catheter body comprising outer and inner tubular members, each having a lumen extending therethrough. The inner tubular member is substantially positioned within the lumen of the outer tubular member. One of the tubular members, such as the outer tubular member, comprises a withdrawal tube for withdrawing bodily fluid from a body vessel for extracorporeal treatment, and the other tubular member, such as the inner tubular member, comprises an infusion tube for infusing the bodily fluid back into the body vessel following extracorporeal treatment of the fluid. At least one of the tubular members includes an expansion member, such as a malecot, for substantially centering the apparatus in a body vessel.

In another embodiment thereof, the invention comprises an assembly for use in treating a bodily fluid. The assembly comprises an outer tubular member having a lumen extending therethrough. The distal end of the outer tubular member has an exit opening, and an expansion member is positioned at said distal end. A dilator having a tapered distal end is selectively receivable in the outer tubular member lumen and removable therefrom. The tapered distal end is sized relative to the outer tubular body exit opening such that a portion of the tapered dilator distal end that has a diameter smaller than the diameter of the exit opening extends distally through the exit opening when the dilator is positioned in the lumen, and a portion of the tapered dilator distal end that has a diameter larger than the diameter of the exit opening abuts but does not pass through the exit opening. The outer tubular member and the dilator are lockingly engageable to maintain the dilator and the outer tubular member in a substantially fixed relative position when the dilator is received in the lumen of the outer tubular member. The expansion member is selectively movable between an expanded condition and a collapsed condition. When the dilator is positioned in the lumen, the expansion member is in a collapsed condition, and when the dilator is removed from the lumen, the expansion member is in an expanded condition.

In yet another embodiment, the present invention comprises a method for treating a bodily fluid. An assembly is provided for transporting the bodily fluid to and from a treatment instrument. The assembly comprises a catheter body having an outer tubular member and an inner tubular member, each of the tubular members having a proximal end and a distal end and a lumen extending therethrough. The inner tubular member is substantially positioned within the lumen of the outer tubular member and extends distally beyond the distal end of the outer tubular member. At least one of the tubular members has an expansion member for substantially centering the assembly in a body vessel. One of the tubular members comprises a withdrawal tube for withdrawing a bodily fluid from the vessel for treatment, and the other tubular member comprises an infusion tube for infusing treated fluid back into the vessel. The distal end of this assembly is inserted into a body vessel. Bodily fluid to be treated is withdrawn from the vessel through the withdrawal tube, and transported to a treatment instrument. Following treatment in the treatment instrument, the treated fluid is transported to the infusion tube for infusion into the vessel through an aperture in the infusion tube.

In a still further embodiment, the present invention comprises a method for treating a bodily fluid. An assembly for transporting the fluid is provided, the assembly comprising an outer tubular member and a dilator. The outer tubular member has a proximal end, a distal end having a distal opening, and a lumen extending therethrough. The outer tubular member further includes an expansion member positioned at its distal end. The dilator is selectively positionable in the lumen of the outer tubular member and removable therefrom. The dilator has a tapered distal end, wherein a portion of the tapered distal end is sized to extend distally through the distal opening of the outer tubular member when the dilator is positioned in the lumen. The expansion member is in a collapsed condition when the dilator is positioned in the lumen. An opening is formed in the body vessel, and the vessel opening is dilated by inserting a distal end of the treatment assembly having the dilator positioned therein into the vessel. The dilator is removed from the dilated opening and from the assembly, thereby allowing the expansion member to attain an expanded condition. An inner member is then inserted in the lumen of the outer tubular member. The inner member has a lumen extending therethrough, and a distal end extending distally beyond the distal end of the outer tubular body. Fluid is withdrawn from the vessel through the lumen of one of the tubular members and transported to a treatment instrument. The fluid is treated in the instrument, and the treated fluid is transported to the other tubular member. The treated fluid is then infused into the vessel through a lumen in the other tubular member and an aperture in the tubular member.

### BRIEF DESCRIPTION OF THE DRAWINGS

FIG. 1 is a side elevational view of one embodiment of the catheter of the present invention;

FIG. 1A is a sectional view of a portion of FIG. 1, showing hub 30 and a proximal portion of inner and outer tubular members 12 and 20;

FIG. 2 is a side elevational view of the inner tubular member of the embodiment of FIG. 1;

FIG. 3 is an enlarged sectional view taken along lines 3-3 of FIG. 1;

FIG. 4 is a side elevational view of the inner tubular member shown in FIG. 2, with the expansion member shown in a compressed state;

FIG. 5 is a side elevational view of an alternative embodiment, wherein both the inner and outer tubular members are provided with expansion members;

FIG. 5A is a fragmented view of the distal end of the catheter of FIG. 5, showing the partial withdrawal of the inner tubular member;

FIG. 6 is a side elevational view of a third embodiment of a catheter assembly;

FIG. 7 is a side elevational view of an outer tubular member of another alternative embodiment of the present invention;

FIG. 8 is a side elevational view of an inner tubular member for the embodiment of FIG. 7;

FIG. 9 is a side elevational view of a catheter apparatus utilizing the outer and inner tubular members shown in FIGS. 7 and 8; and

FIG. 10 is a side elevational view of the apparatus of FIG. 9, wherein a pigtail catheter has been introduced to replace the inner tubular member.

### DETAILED DESCRIPTION OF THE DRAWINGS AND THE PRESENTLY PREFERRED EMBODIMENTS

For purposes of promoting an understanding of the present invention, reference will now be made to the embodiments illustrated in the drawings, and specific language will be used to describe the same. It is nevertheless to be understood that no limitation of the scope of the invention is thereby intended, the proper scope of the invention being indicated by the claims appended below and the equivalents thereof. The figures are not all drawn to the same scale to avoid obscuring the details of the finer structures. The following detailed description of the preferred embodiments will make clear the preferred arrangement, size relationships and manner of using the components shown herein.

The present invention comprises a multi-lumen catheter for use in the extracorporeal treatment of bodily fluids. In a preferred embodiment, the invention comprises a dual lumen catheter for extracorporeal use, such as for use in a hemodialysis procedure. In addition to hemodialysis, the catheter can be used for other extracorporeal fluid treatments in which a body fluid is withdrawn from the body, subjected to a treatment process, and thereafter returned to the body. Pheresis and hemofiltration are non-limiting examples of such procedures.

The dual lumen catheter of the preferred embodiment includes two elongated tubular members, each having a lumen therethrough. At least one of the tubular members includes an expansion member, such as a malecot cut in the wall of the tubular member. The expansion member expands the effective diameter of the catheter at the point of the expansion member, and thereby assists in centering the main portion of the catheter in the centermost portion of the vessel. Fluid passing through the center area of a body vessel generally flows at a higher flow rate than fluid passing near the vessel walls. Formation of fibrin and other types of blockages are less likely to occur on catheters and other medical devices when the device is positioned in a high flow area, such as in the center area of the vessel. In addition, the structure of the expansion member will generally also include open areas. These open areas also serve to further enhance the flow rate of fluids into or out of the catheter, by expanding the ingress or egress areas of the catheter through which the fluid may flow. Although the description herein specifically makes reference to a dual lumen catheter, those skilled in the art will appreciate that additional lumens can be provided on the catheter assembly for other purposes well known in the art, such as the administration of medicaments and the like.

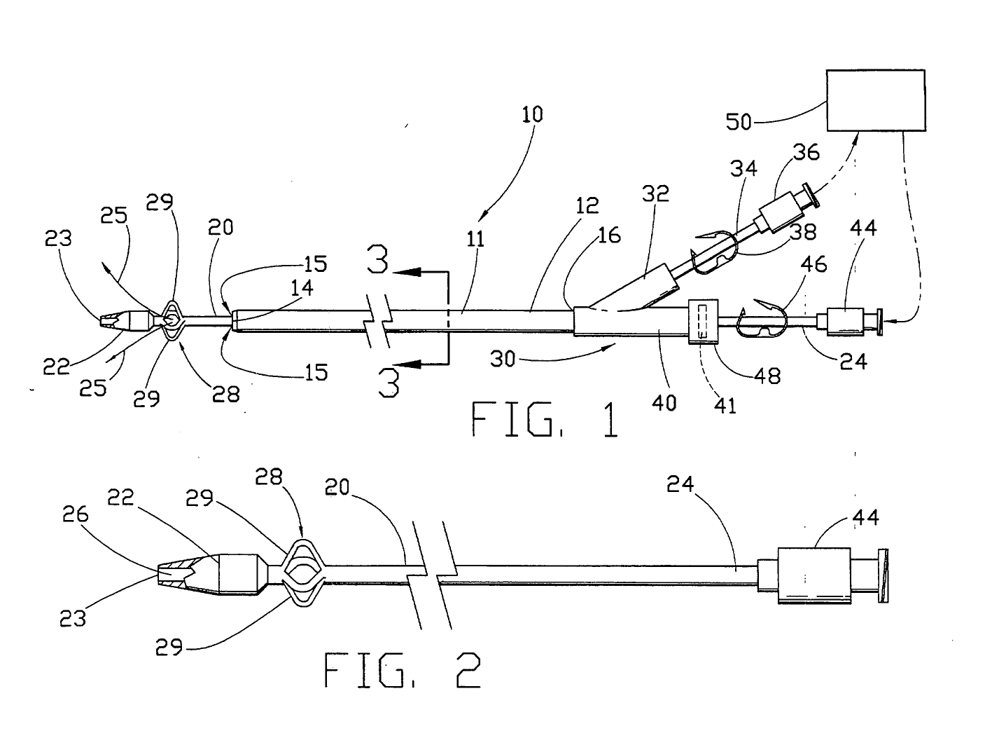

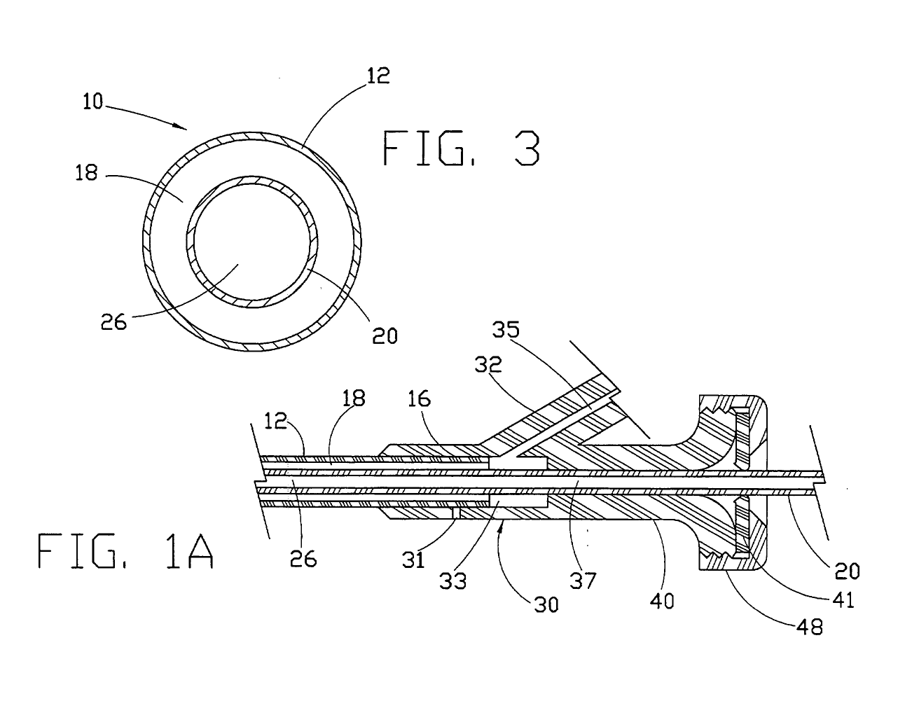

FIG. 1 is a side elevational view of one embodiment of a catheter assembly 10 according to the present invention. In the following discussion, the terms “proximal” and “distal” will be used to describe the axial ends of the apparatus, as well as the axial ends of various component features. The term “proximal end” refers to the end of the catheter assembly (or component) that is closest to the operator during use of the assembly. The term “distal end” refers to the end of the assembly (or component) that is inserted into the patient, or that is closest to the patient. In the orientation of catheter assembly 10 and each of its component features shown in the figures herein, the proximal end is to the right, while the distal end is to the left.

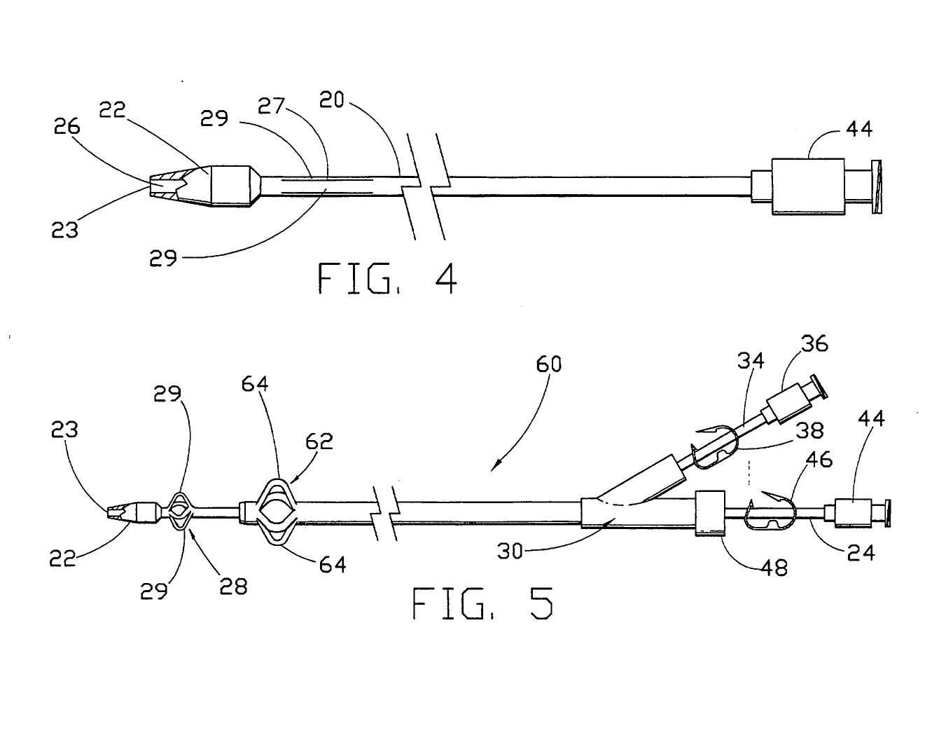

Catheter assembly 10 includes a catheter body 11. Catheter body 11 comprises an outer elongated tubular member 12 having a distal end 14, a proximal end 16 and a lumen 18 extending therethrough (FIG. 3). Catheter body 11 also includes a removable inner elongated tubular member 20 having a distal end 22, a proximal end 24, and a lumen 26 extending therethrough. For clarity, FIG. 2 shows the inner tubular member separate from outer tubular member 12. When catheter assembly 10 is fully assembled as shown in FIG. 1, the main shaft portion of inner tubular member 20 is disposed in lumen 18 of the outer tubular member. FIG. 3 shows the coaxial orientation of outer and inner tubes 12, 20 and their respective lumens 18, 26. In this embodiment, inner tubular member 20 also includes an expansion member 28, preferably disposed in the vicinity of distal end 22. Expansion member 28 will be further described hereinafter.

In the preferred embodiment shown in FIG. 1, outer tubular member 12 comprises the fluid withdrawal tube. Preferably, distal end 14 of outer tubular member 12 terminates proximal to distal end 22 of inner tubular member 20, as shown in the figure. Arrows 15 are provided on FIG. 1 to indicate the direction of arterial flow as blood passes from the body vessel (not shown), into lumen 18 of outer tubular member 12. The withdrawn arterial blood is preferably drawn through lumen 18 under negative pressure, past outer tube proximal end 16 and into hub 30 for further transport and treatment in a manner to be described.

Inner tubular member 20 comprises the fluid infusion, or return, tube. Although outer tubular member 12 and inner tubular member 20 have been designated in this embodiment as the respective withdrawal and infusion tubes, this designation may be reversed, if desired. Arrows 25 are provided in FIG. 1 to indicate the direction of venous flow of treated blood as it is infused, or returned, to the body vessel. Blood is preferably infused to the vessel under positive pressure, through the openings defined by rib members 29, as well as through exit opening 23 at distal end 22 of inner tubular member 20. Preferably, distal end 22 comprises an atraumatic distal tip.

Inner tubular member 20 includes expansion member 28. Expansion member 28 is preferably provided at the distal end of inner tubular member 20, although it need not necessarily be situated at this end. In FIGS. 1 and 2, expansion member 28 is shown in its expanded state. In the view of FIG. 4, expansion member 28 is shown in a compressed state. The expansion member shown in these figures is a malecot cut. Such expansion members are well known in the art, and are shown, e.g., in U.S. Pat. No. 4,808,163, incorporated by reference herein. A malecot comprises expandable rib members 29 that are formed by cutting spaced slits 27 (FIG. 4) in the distal end of tubular member 20.

Preferably, sufficient slits are cut into tubular member 20 to form four rib members 29, spaced 90° along the circumference of the inner tubular member. More, or fewer, ribs can be substituted without departing from scope of the invention. During formation of rib members 29, a spacer can be inserted inside the expansion member at the area of the slits 27 to stretch the expandable member such that it takes the desired configuration. This portion of tubular member 20 is then treated, such as by passing the catheter through steam. The catheter body is then cooled in the expanded configuration, and the spacer is removed. The heat treatment provides the catheter body with a memory, so that the natural “at rest” position of the expansion member 28 becomes the expanded condition shown in FIGS. 1 and 2.

In this embodiment, inner tubular member 20 is removable from lumen 18 of outer tubular member 12. When the distal portion of inner tubular member 20 (including expansion member 28) is disposed within lumen 18, such as during insertion and withdrawal of the apparatus, rib members 29 of inner tubular member 20 are in the compressed state shown in FIG. 4. As expansion member 28 passes through lumen 18 to the position shown in FIG. 1 during insertion, the memory formed in rib members 29 causes the slitted (ribbed) sections to radially splay out in well-known fashion to the configuration shown in FIGS. 1 and 2.

The expanded configuration slightly shortens the axial length of inner tubular member 20. By increasing the effective diameter of inner tubular member 20 at the site of expansion member 28, tubular member 20 acts to substantially center catheter assembly 10 in the body vessel, thereby inhibiting blockage of the ports. The configuration of expansion member 28 also defines multiple infusion openings, e.g., between rib members 29, thereby enhancing the flow rate of blood back into the vessel.

Hemodialysis catheters are prone to blockage, which can render the device unusable. Utilization of a removable inner tubular member such as that described enables the physician to replace a blocked tube with a new one. As a result, the patient is benefited since the useful lifetime of a catheter can be extended.

In the preferred embodiment shown, catheter assembly 10 also includes a side-arm fitting, such as hub 30. Hub 30 branches in the proximal direction into two legs 32, 40. As shown in FIG. 1A, outer tubular member 12 is received in a cylindrical chamber 33 in hub 30 in conventional fashion, such as by gluing distal end 16 of inner tubular member 12. Hub 30 may be provided with a glue hole 31 for inserting glue into chamber 33. Alternatively, outer tubular member 12 can be engaged with hub 30 in other conventional ways well known in the art. Hub 30 further includes passageway 35 for transport therethrough of blood withdrawn from the body vessel, and passageway 37 for the return of treated blood back to the vessel.

Preferably, infusion, or return, hub leg 40 includes a conventional self-closing valve, such as hemostatic valve 41. In the embodiment shown in FIG. 1A, hemostatic valve 41 comprises a conventional elastomeric valve disk having an opening to permit passage therethrough of inner tubular member 20. Elastomeric valve disk 41 is self-conforming to the outer surface of the inner tubular member. When an inner tubular member is to be replaced, it may be simply withdrawn in the proximal direction through valve 41, thereby causing the opening in elastomeric valve 41 to close, or seal. A new inner tubular member may then be inserted through valve 41 in the same manner, at which time the opening in valve 40 conforms to the dimensions of the new tubular member. As an alternative to a hemostatic valve, other conventional valves or seals can be utilized, such as a Touhy-Borst or adjustable compression seal around the inner tubular member. This type of seal could also serve to lock the inner and outer tubular members together if desired.

In a preferred embodiment, catheter assembly 10 also includes at least one extension tube, such as extension tube 34, as well as connectors 36, 44. Extension tube 34 is engaged with the proximal end of leg 32. A luer lock or other suitable connector 36 is engaged with the proximal end of extension tube 34. Connector 36 engages in mating relationship with a connector associated with an ingress opening of a treatment instrument 50, such as a dialyzer, for establishing a flow path of blood to the dialyzer. Conventional clamp 38 may be provided for selectively opening and closing extension tube 34.

Proximal end 24 of inner tubular member 20 is engaged with the proximal end of hub leg 40. A luer lock or other suitable connector 44 is engaged with proximal end 24. Connector 44 engages in mating relationship with a connector associated with an egress opening of the dialyzer. Dialyzer 50 and its ingress and egress openings are shown schematically in FIG. 1. Conventional clamp 46 may be provided for selectively opening and closing the distal end of tube 20. Alternatively, an extension tube may be engaged with the proximal end of inner tubular member 20, and can be utilized with clamp 46 in the same manner as extension tube 34. Preferably, a screw cap 48 or like device can be provided for securing hemostatic valve 41 in place.

Extension tube 34 can be formed to have virtually any length, however it should be long enough to receive clamp 38. Preferably, extension tube 34 is relatively soft and flexible so that it can be easily manipulated and closed by the pressure exerted by the clamp. The clamps function in the nature of valves to control the flow of blood between the dialyzer and the catheter. Those skilled in the art will appreciate that the extension member shown in FIG. 1 need not be a discrete element as shown, and may comprise an elongated portion of the side arm tube. Thus, as stated, the presence of one or more extension tubes is optional.

Use of the catheter assembly 10 of FIG. 1 in a hemodialysis procedure will now be described. Initially, the catheter assembly must be introduced into the vessel. Suitable percutaneous techniques for insertion of catheter assemblies into body vessels are well known in the medical arts, and are in widespread use. Perhaps the most widely-utilized technique, and the technique favored herein, is the well-known Seldinger technique. In the Seldinger technique, an injection is made into the vessel interior with a needle, and a wire guide is inserted into the vessel through a bore in the needle. The needle is withdrawn, and an introducer sheath, preferably a splittable sheath, such as a PEEL-AWAY® sheath, available from Cook Incorporated, of Bloomington, Ind., is introduced over the wire guide. The catheter assembly is then introduced into the vessel via the introducer sheath and over the wire guide. The wire guide and the sheath are removed in conventional fashion, leaving the distal end of catheter assembly 10 in the body vessel.

Once catheter assembly 10 has been inserted into a body vessel, sufficient negative pressure is created to commence withdrawal of blood from the vessel, such as by activation of dialyzer 50. Blood is withdrawn from the vessel in the direction of arrows 15 of FIG. 1, and enters distal end 14 of outer tubular member 12. The blood is then drawn through lumen 18 in the proximal direction, and passes into dialyzer 50 by way of a flow path that includes hub leg 32, extension tube 34 and connector 36. The withdrawn blood is then subjected to treatment in dialyzer 50 in conventional fashion. Following treatment, the cleansed blood is returned to the body vessel by way of a flow path that includes connector 44, hub leg 40 (including hemostatic valve 41) and lumen 26. The cleansed blood then re-enters the vessel through exit opening 23 and the openings formed between expanded rib members 29 of the expansion member. By infusing blood through expanded rib members as well as through the exit opening, the flow rate of blood may be kept to a minimum velocity, since the blood does not need to pass through any restriction. Passing blood through such a restriction would undesirably increase its velocity, which can result in cell damage, such as hemolysis.

FIG. 5 is a side elevational view of a second embodiment of a catheter assembly 60. Features in common with the embodiment of FIGS. 1-4 have the same reference numerals as in the previous embodiment. In the embodiment of FIG. 5, outer tubular member 12 is also provided with an expansion member 62. Expansion member 62 preferably also comprises rib members 64, and can be formed in the same manner as expansion member 28, and to have the same, or different, dimensions. Expansion member 62 is in its compressed state when it is disposed within the inner lumen of an introducer sheath, and expands to the configuration shown in FIG. 5 when inserted into the vessel and following withdrawal of the introducer sheath.

Providing a second expansion member 62 as shown further enhances the ability of catheter assembly 10 to remain centered in the vessel, and in addition, provides enhanced flow area for blood being withdrawn from the vessel to enter lumen 18. Providing a greater flow area in this manner further diminishes the possibility of blockage of the withdrawal port. In this embodiment, the inner tubular member is also removable, as in the previous embodiment. FIG. 5A shows the position of distal end 22 of inner tubular member 30 during withdrawal of this member through the lumen of outer tubular member 12. Inner tubular member distal end 22 can be provided with a cylindrical portion 42 that is sized to pass through the lumen of the outer tubular during withdrawal.

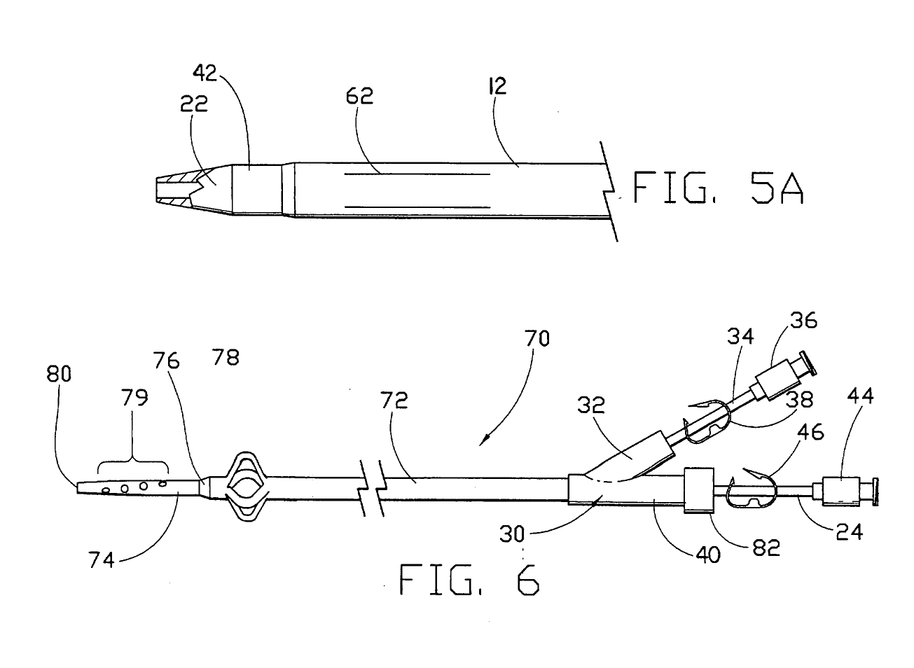

FIG. 6 is a side view of a third embodiment of a catheter assembly 70 for extracorporeal treatment of bodily fluids. Features in common with the embodiment of FIGS. 1-5 have the same reference numerals as in the previous embodiment. In this embodiment, outer tubular member 72 and inner tubular member 74 are bonded together at a bonding site 76 near the distal end of catheter assembly 70. Thus, in this embodiment, the inner tubular member is not removable from the assembly. An expansion member 78 is provided on outer tube 72 to center assembly 70 in the vessel, and to assist in withdrawal of fluid from the vessel through hub 30, extension tube 34 and connector 36 as before. Following treatment, the fluid is returned to inner tubular member 74 through connector 44 and hub 30, in a manner similar to that of the previous embodiments. The cleansed blood then reenters the body vessel through side infusion apertures 79, spaced along the outer surface of tubular member 74, and through exit opening 80. Side infusion apertures are preferably spaced in generally spiral fashion along the distal end of inner tube 74. Virtually any number of apertures 79 may be provided, and the apertures may be aligned in virtually any configuration along the distal end of the inner tube to accomplish their purpose of infusing cleansed blood back into the vessel.

With this embodiment, catheter assembly 70 may be introduced over a wire guide. A separate introducer sheath is not necessary. When inserted, the expansion member 78 is in its compressed condition. To activate expansion member 78 following introduction of catheter assembly 70 into a vessel, connector portion 44 is pulled back in a proximal direction, and locked in place with a suitable locking mechanism 82, such as a friction O-ring. This action maintains the expansion member in its expanded condition as shown in the figure.

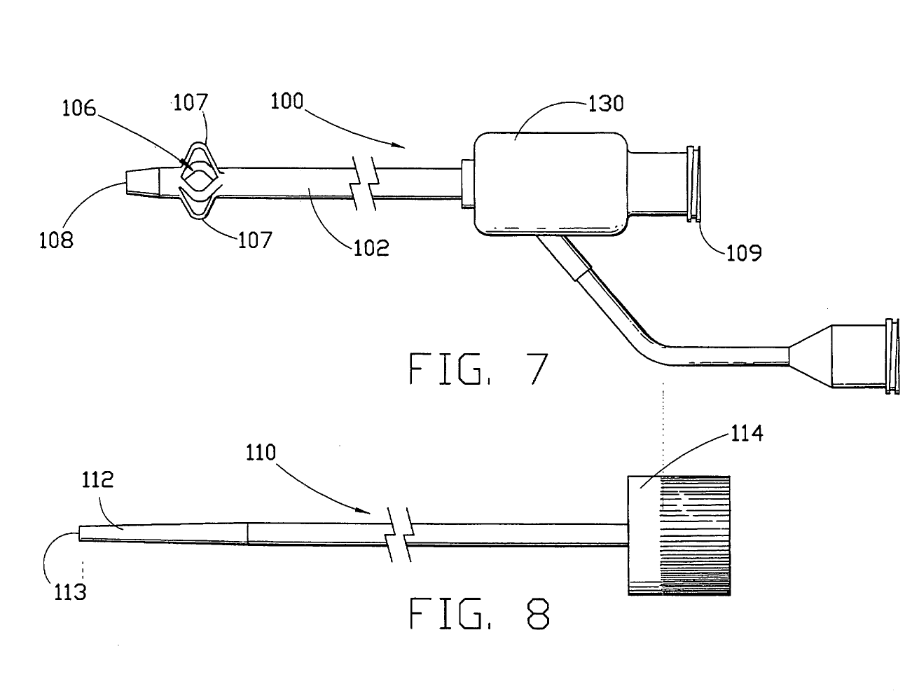

Another alternative embodiment of a catheter assembly 100 according to the present invention is shown in FIGS. 7-10. In this embodiment, catheter assembly 100 comprises tubular member 102 having distal expansion member 106. Expansion member 106 has a natural “at rest” expanded configuration, such as the malecot shown in FIG. 7 having rib members 107. A dilator 110 (FIG. 8) is insertable into the lumen of tubular member 102 at the proximal end of member 102 through an aperture in connector 109. Dilator 110 has a tapered distal end 112, terminating at distal tip 113. Preferably, distal end 112 tapers such that distal tip 113 has a diameter substantially the same as, or slightly larger than, the diameter of a wire guide passing therethrough.

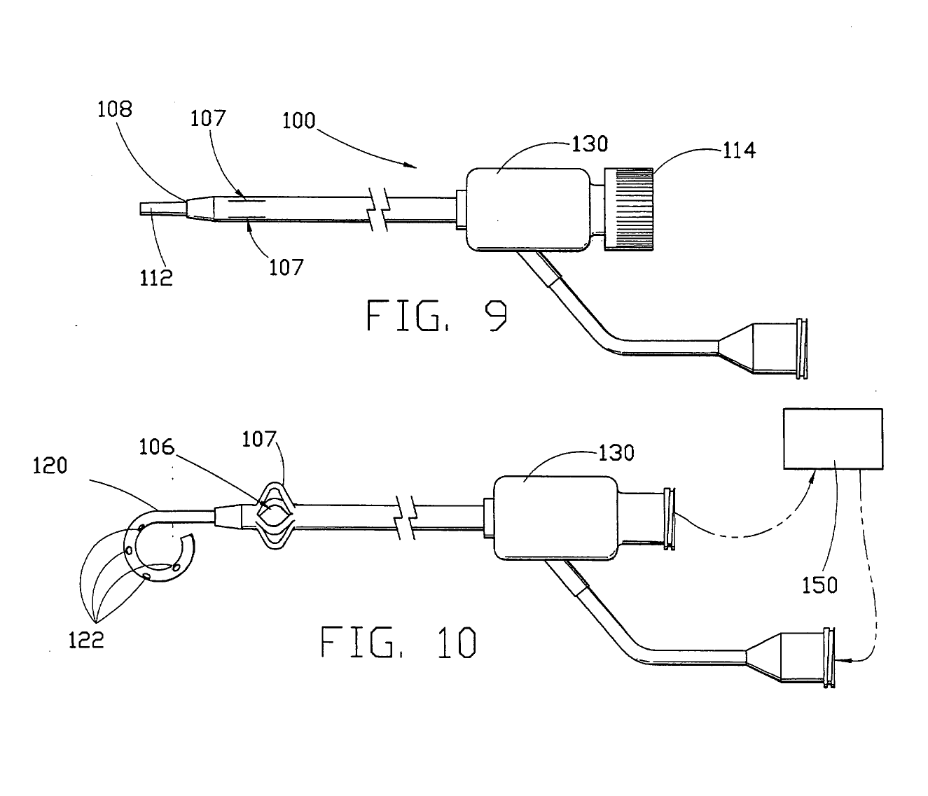

When dilator 110 is fully inserted into the lumen of tubular member 102, as shown in FIG. 9, a small diameter portion of distal end 112 extends through tubular member exit opening 108. The outer diameter of dilator distal end 112 is tapered and sized such that only this small diameter portion of tapered distal end 112 can extend through exit opening 108. As dilator 110 is advanced in the distal direction through the lumen of tubular member 102 such that the small diameter portion of distal end 112 extends through the exit opening, a larger diameter portion of tapered distal end 112 engages the inner circumference of tubular member 102 that surrounds the exit opening 108, but cannot pass therethrough. Further distal advancement of dilator 110 slightly stretches this inner circumference around exit opening 108. As a result, expansion member 106 is stretched in the distal direction, and thereby collapses from its expanded condition shown in FIG. 7 to a collapsed condition shown in FIG. 9. In the collapsed condition, rib members 107 are substantially flattened out along the outer surface of tubular member 102. A connector 114 at the proximal end of dilator 110 may be used to lock dilator 110 to the proximal end of tubular member 102 via mating tubular member connector 109 to maintain this collapsed condition. Tubular member 102 and dilator 110 are shown in the locked condition in FIG. 9. Suitable locking mechanisms, such as luer locks, are well known in the art, and the skilled artisan can readily select a suitable mechanism for a particular use.

When assembly 100 is in the closed, or locked, configuration of FIG. 9, the assembly can be introduced into the vessel over a conventional wire guide. In this case, assembly 100 also functions as an introducer sheath, and a separate introducer is therefore not necessary. When a wire guide is used, dilator 110 and dilator cap 114 include a passageway therethrough to allow for passage of the wire guide. After the assembly has been properly positioned in the vessel, the dilator can be disengaged from the tubular member and removed from the assembly, thereby allowing the malecot to return to its natural expanded configuration shown in FIG. 7. An inner catheter member can then be placed within the lumen of tubular member 102.

In the embodiment shown in FIG. 10, a conventional pigtail catheter 120 is shown as the removable inner member. Alternatively, an inner tubular member of the type used in previous embodiments may be substituted for the pigtail catheter with only minor modification. As with the previous embodiments, the expansion member assists in centering catheter assembly 100 in the vessel. Blood is withdrawn from the vessel through exit opening 108 and through the openings between rib members 107 of the expansion member.

The withdrawn blood passes through the lumen of tubular member 102, and is transported under negative pressure to dialyzer 150 (FIG. 10). The cleansed blood is then returned through the central lumen of pigtail catheter 120. Pigtail catheter 120 has a plurality of side ports 122 at its distal end, through which the blood is infused into the vessel. In many ways, the use of a pigtail catheter is analogous to that of an infusion malecot. Since it has a plurality of side ports, there is an increased likelihood that at least some of the side ports will remain unobstructed, even if other ports become blocked. In addition, some or all of the side ports remain in the vicinity of the radial center of the vessel. In this position, the side ports are not subject to obstruction by being pressed up against the vessel wall. Since the flow of blood is fastest near the center of the vessel, blood is less likely to clot on the surface of a device that is positioned in a high flow area, as compared to areas of lower flow such as the area near the vessel wall.

As with the previous embodiments, blood may flow between the apparatus 100 and the dialyzer in conventional fashion through any desired arrangement of extension tubes, connectors, luer locks etc. (not shown).

Although the embodiments in the figures show specific arrangements of expansion members on inner and outer catheter tubular members, the invention is not limited to those embodiments shown. Rather, expansion members can be placed on any combination of either, or both, expansion members. For multi-lumen catheters having more than two tubular members, expansion members can likewise be placed on any, or all, of the tubular members. Since the withdrawal of fluid is likely to occur under negative pressure, the formation of fibrin may be more problematic at the withdrawal port, thus perhaps making the withdrawal tube a more likely candidate for inclusion of an expansion member. The infusion of fluid back into the body vessel is likely to occur under positive pressure at the infusion port, so that, all other factors equal, it may not be as important to include an expansion member on the infusion tube as on the withdrawal tube. Nevertheless, in a particular case benefits may be obtained using an expansion member even in a positive pressure situation, particularly in view of the vessel centering function of the expansion member. Therefore, the present invention contemplates use of an expansion member in either a positive or negative pressure situation, or both.

To further enhance fluid flow, one or more apertures or side ports can also be placed on any of the tubular structures that make up the catheter, regardless of whether the particular tubular structure also includes an expansion member.

It should be noted that in each of the figures herein, the expansion member is positioned near the distal end of the tubular member. Although this is the preferred placement of the expansion member, the invention is not so limited. Rather, the expansion members can be placed anywhere along the longitudinal surface of the tubular member, and it is expected that some benefit may be obtained thereby.

If desired, various components of the catheters and assemblies described herein, such as the tubular members and the dilator, can be impregnated or coated with antimicrobial agents to minimize the risk of bacterial colonization of the catheter, and catheter-related bacteremia during use. An example of an antimicrobial combination that has been shown to be an effective antimicrobial composition in percutaneous devices is the combination of antimicrobials minocycline and rifampin. Alternatively, other well-known antimicrobials may be substituted for minocycline and rifampin, which antimicrobials need not necessarily be utilized in combination.

Although the embodiment described hereinabove discusses the extracorporeal treatment of blood, those skilled in the art will appreciate that the catheter will have use in the treatment of other body fluids, such as cerebrospinal fluid.

The use of catheter of the present invention is beneficial to a patient undergoing a procedure that requires extracorporeal treatment of a fluid, because it minimizes the number of times that such a catheter must be replaced. Each time that a catheter is replaced, a new site must be found for the new catheter. Eventually, a patient will run out of acceptable placement sites. Thus, the use of a catheter that centers the catheter in high flow areas of a vessel lessens the possibility of port blockage. In addition, the use of a removable inner tubular member enables the patient to extend use at a particular placement site even though an inner tube may have become blocked, and therefore unusable. As a result of these benefits, there is a lesser likelihood that a patient will use up all of the acceptable sites for insertion of the catheter.

While this invention has been particularly shown and described with reference to preferred embodiments thereof, it will be understood by those skilled in the art that various changes in form, details, and composition of the various components may be made therein without departing from the spirit and scope of the invention, and any such variations are considered to be within the scope of the invention. Those skilled in the art may recognize or be able to ascertain using no more than routine experimentation, many equivalents to the specific embodiments of the invention described specifically herein, which equivalents are intended to be encompassed in the scope of the invention.

1. A catheter assembly for use in extracorporeal treatment of a bodily fluid, comprising:
a catheter body, said catheter body comprising an outer tubular member having a proximal end and a distal end and a lumen extending therethrough, and an inner tubular member having a proximal end and a distal end and a lumen extending therethrough; said inner tubular member substantially positioned within the lumen of said outer tubular member; wherein at least one of said tubular members has an expansion member.

2. The catheter assembly of claim 1, wherein said expansion member is positioned at a distal end of said at least one tubular member.

3. The catheter assembly of claim 2, wherein said outer tubular member comprises a withdrawal tube for withdrawing said bodily fluid from a body vessel for extracorporeal treatment, and said inner tubular member comprises an infusion tube for infusing said bodily fluid into said body vessel following said extracorporeal treatment.

4. The catheter assembly of claim 3, wherein said expansion member is positioned at a distal portion of said inner tubular member.

5. The catheter assembly of claim 4, wherein said expansion member comprises a plurality of expandable rib members.

6. The catheter assembly of claim 2, wherein an expansion member is positioned at a distal portion of each of said tubular members.

7. The catheter assembly of claim 2, wherein said inner tubular member is removably mounted to said catheter body.

8. The catheter assembly of claim 2, wherein said outer tubular member is bonded to said inner tubular member at a distal portion of said inner tubular member.

9. The catheter assembly of claim 8, wherein said expansion member is disposed at a distal portion of said outer tubular member, and wherein said inner tubular member includes a plurality of infusion apertures.

10. The catheter assembly of claim 3, comprising a hub member for engagement with respective proximal ends of said outer and inner tubular members, and further comprising an extension member having proximal and distal ends, said extension member engaged at said distal end with said hub member to receive withdrawn bodily fluid and engaged at said proximal end with an ingress opening to an instrument for use in said extracorporeal treatment.

11. The catheter assembly of claim 10, further comprising a second extension member having proximal and distal ends, said second extension member engaged at said proximal end with an egress opening for said instrument and at said distal end with said hub member for transporting treated fluid to said infusion tube for infusion into said body vessel.

12. The catheter assembly of claim 11, wherein at least one of said extension members includes a connector for engagement with a mating connector of said instrument.

13. The catheter assembly of claim 11, wherein said treated fluid is infused to said vessel through an exit opening at said distal end of said inner tubular member, and through openings in said expansion member.

14. The catheter assembly of claim 10, wherein said hub member further comprises a hemostatic valve.

15. The catheter assembly of claim 9, wherein said expansion member is selectively movable between an expanded condition and a compressed condition.

16. The catheter assembly of claim 15, further comprising a locking mechanism for selectively locking said expansion member in said expanded condition or said compressed condition.

17. The catheter assembly of claim 16, wherein said locking mechanism comprises a locking member located at the proximal end of each of said outer and inner tubular members, said locking members being selectively engageable in locking relationship.

18. The catheter assembly of claim 17, wherein said expansion member is in said compressed condition when said locking members are engaged in said locking relationship.

19. The catheter assembly of claim 4, wherein said inner tubular member is movable in said catheter body relative to said outer tubular member between a first position wherein said inner tubular member distal end is disposed within the lumen of the outer tubular member and a second position wherein said inner tubular member distal end extends axially beyond said outer tubular member distal end, said expansion member being in a compressed condition when said inner tubular member is in said first position, and in an expanded condition when said inner tubular member is in said second position.

20. The catheter assembly of claim 3, comprising a hub member for engagement with said proximal end of said outer tubular member and said proximal end of said inner tubular member, and further comprising a valve in said hub member.

21. The catheter assembly of claim 20, wherein said valve comprises an elastomeric valve having an opening therethrough for passage of said inner tubular member, and wherein said inner tubular member is removable from said catheter assembly through said valve opening, said valve opening conforming to an outside surface of said inner tubular member such that said valve member is in an open position when said inner tubular member is positioned in said catheter assembly, and in a closed position when said inner tubular member is removed from said assembly.

22 The catheter assembly of claim 21, further comprising a replacement inner tubular member for replacing a removed inner tubular member.

23. The catheter assembly of claim 21, further comprising a removable cap member for permitting selective positioning of said inner tubular member in said catheter assembly and removal therefrom.

24. The catheter assembly of claim 1, wherein at least a portion of said catheter assembly includes an antimicrobial coating.

25. An assembly for use in treating a bodily fluid, comprising:
an outer tubular member having a proximal end and a distal end and a lumen extending therethrough, said distal end having an exit opening, said exit opening having a diameter, said outer tubular member including an expansion member positioned at said distal end, and further including a locking member;
a dilator, said dilator having a proximal end and a tapered distal end, said dilator being selectively receivable in said outer tubular member lumen and removable therefrom, said tapered distal end being sized relative to said outer tubular body exit opening such that a portion of said tapered dilator distal end having a diameter smaller than the diameter of said exit opening extends distally through said exit opening when said dilator is received in said lumen, and a portion of said tapered dilator distal end having a diameter larger than the diameter of the exit opening abuts but does not pass through said exit opening; said dilator further including a locking member, said dilator locking member engageable with said outer tubular member locking member to maintain said dilator and outer tubular member in a substantially fixed relative position when said dilator is received in said lumen.

26. The assembly of claim 25, wherein said locking members are located at respective proximal portions of said outer tubular member and said dilator.

27. The assembly of claim 25, wherein said expansion member is movable between an expanded condition and a collapsed condition, and wherein said expansion member is in said collapsed condition when said dilator is received in said lumen and said dilator large diameter portion abuts and exerts a force against said exit opening.

28. The assembly of claim 27, wherein said outer tubular member lumen comprises a first lumen, said assembly further comprising an inner member sized and shaped to be received in said first lumen when said dilator is removed therefrom, said inner member having a proximal end, a distal end and a lumen extending therethrough, said inner member lumen comprising a second lumen, said inner member having at least one aperture at said distal end, said inner member distal end extending distally beyond said outer tubular body distal end.

29. The assembly of claim 28, wherein said outer tubular member comprises a withdrawal member for withdrawing said bodily fluid from a body vessel through said first lumen, and said inner member comprises an infusion member for infusing said bodily fluid into said body vessel through said second lumen.

30. The assembly of claim 29, wherein said inner member comprises a pigtail catheter, said pigtail catheter having a plurality of apertures at said distal end for infusing said fluid into said vessel.

31. The assembly of claim 29, wherein said inner member comprises a tube, said tube having at least one aperture at said distal end.

32. The assembly of claim 31, wherein said inner member comprises an expansion member at said distal end.

33. A method for treating a bodily fluid, comprising:
providing an assembly for transporting said bodily fluid, said assembly comprising a catheter body having an outer tubular member and an inner tubular member, each of said outer tubular member and said inner tubular member having a proximal end and a distal end and a lumen extending therethrough, said inner tubular member substantially positioned within the lumen of said outer tubular member and extending distally beyond the distal end of said outer tubular member; at least one of said outer and inner tubular members having an expansion member for substantially centering said assembly in a body vessel, one of said tubular members comprising a withdrawal tube for withdrawing a bodily fluid from said vessel for treatment, and the other of said tubular members comprising an infusion tube for infusing treated fluid into said vessel;
inserting a distal end of said assembly into said vessel;
withdrawing said bodily fluid to be treated from said vessel through said withdrawal tube;
transporting said withdrawn fluid to a treatment instrument;
treating said fluid in said treatment instrument;
transporting said treated fluid to said infusion tube; and
infusing said treated fluid into said vessel through an aperture in said infusion tube.

34. The method of claim 33, wherein said expansion member is disposed at a distal portion of said inner tubular member.

35. The method of claim 34, wherein an expansion member is disposed at a distal portion of each of said tubular members.

36. The method of claim 34, wherein said expansion member comprises a malecot.

37. The method of claim 33, wherein said outer tubular member comprises said withdrawal tube, and said inner tubular member comprises said infusion tube.

38. The method of claim 33, wherein said inner tubular member is removably mounted to said assembly.

39. The method of claim 33, wherein said outer tubular member is bonded to said inner tubular member at a distal portion of said inner tubular member.

40. The method of claim 39, wherein said expansion member is disposed at a distal portion of said outer tubular member, and wherein said inner tubular member includes a plurality of infusion apertures.

41. The method of claim 33, further comprising the steps of injecting a needle into said vessel, inserting a wire guide into said vessel through a bore of said needle, withdrawing said needle, and thereafter inserting said distal end of said assembly into said vessel over said wire guide.

42. The method of claim 41, wherein the assembly is inserted into said vessel in a locked condition, wherein said outer and inner tubular members are in a fixed position relative to each other, and said expansion member is in a collapsed condition.

43. The method of claim 42, wherein said outer and inner tubular members are locked at their respective proximal ends.

44. The method of claim 42, further comprising the step of unlocking said tubular members following insertion into the vessel, to thereby activate said expansion member.

45. The method of claim 33, wherein said instrument is a dialyzer and said treating step comprises cleansing blood in said dialyzer.

46. The method of claim 33, wherein at least a portion of said assembly is coated with an antimicrobial agent.

47. A method for treating a bodily fluid, comprising:
providing a treatment assembly, said assembly comprising an outer tubular member and a dilator, said outer tubular member having a proximal end, a distal end having a distal opening, and a lumen extending therethrough, said outer tubular member including an expansion member positioned at said distal end; said dilator selectively positionable in said lumen and removable therefrom, said dilator having a tapered distal end wherein a portion of said tapered distal end is sized to extend distally through said distal opening of said outer tubular member when said dilator is positioned in said lumen, said expansion member being in a collapsed condition when said dilator is positioned in said lumen;
forming an opening in a body vessel;
dilating said vessel opening by inserting a distal end of said treatment assembly having said dilator positioned therein into said vessel;
removing said dilator from said dilated opening and said assembly, thereby causing said expansion member to attain an expanded condition;
inserting an inner member in said lumen of said outer tubular member, said inner member having a lumen extending therethrough, and having a distal end extending distally beyond said outer tubular body distal end, said inner member distal end having at least one aperture therein;
withdrawing fluid from said vessel through the lumen of said outer tubular member;
transporting said withdrawn fluid to a treatment instrument, and treating said fluid in said instrument;
transporting said treated fluid to said inner tubular member; and
infusing said treated fluid into said vessel through a lumen in said inner tubular member and an aperture in said infusion tube.

48. The method of claim 47, wherein said inner member comprises a pigtail catheter.

49. The method of claim 47, wherein said instrument is a dialyzer and said treating step comprises cleansing blood in said dialyzer.

50. The method of claim 47, wherein at least a portion of said assembly is coated with an antimicrobial agent.
